(19) United States
(12) Patent Application Publication
(10) Pub. No.: US 2006/0142392 A1
(43) Pub. Date: Jun. 29, 2006

(54) Process for preparing (2s)-3-(4-{2-[amino]-2-oxoethoxy}phenyl)-2-ethoxypropanoic acid derivatives

(75) Inventors: Carl-Johan Aurell, Sodertalje (SE); Anna Minidis, Sodertalje (SE); Esmail Yousefi-Salakdeh, Sodertalje (SE)

Correspondence Address: FISH & NEAVE IP GROUP;ROPES & GRAY LLP, ONE INTERNATIONAL PLACE, BOSTON, MA, 02110-2624, US

(73) Assignee: AstraZeneca AB, Sodertalje (SE)

(21) Appl. No.: 10/560,764

(22) PCT Filed: Jun. 16, 2004
(86) PCT No.: PCT/SE04/00966

§ 371 Date: Dec. 13, 2005

(30) Foreign Application Priority Data
Jun. 18, 2003 (GB) 0314134.8

(51) Int. Cl. A61K 31/195; C07C 237/30

(52) U.S. Cl. 514/563; 562/450

(57) ABSTRACT

The present invention provides a process for the preparation of a compound of formula I 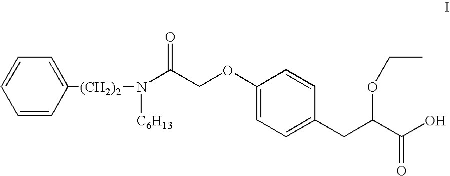
in which a compound of formula II 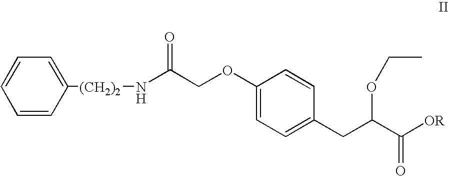 in which R is H or OR represents a protecting group for a carboxylic hydroxy group is reacted with a compound of formula III
C₆H₁₃X III wherein X is a leaving group, in the presence of a base in the presence of an inert solvent at a temperature in the range −25° C. to 150° C. and optionally, when OR represents a protecting group, removal of the protecting group.

### FIELD OF THE INVENTION

The present invention relates to processes for preparing certain (2S)-3-(4-{2-[amino]-2-oxoethoxy}phenyl)-2-ethoxypropanoic acid derivatives.

### BACKGROUND OF THE INVENTION

The metabolic syndrome including type 2 diabetes mellitus, refers to a cluster of manifestations including insulin resistance with accompanying hyperinsulinaemia, possibly type 2 diabetes mellitus, arterial hypertension, central (visceral) obesity, dyslipidaemia observed as deranged lipoprotein levels typically characterised by elevated VLDL (very low density lipoproteins), small dense LDL particles and reduced HDL (high density lipoprotein) concentrations and reduced fibrinolysis.

Recent epidemiological research has documented that individuals with insulin resistance run a greatly increased risk of cardiovascular morbidity and mortality, notably suffering from myocardial infarction and stroke. In type 2 diabetes mellitus atherosclerosis related conditions cause up to 80% of all deaths.

In clinical medicine there is awareness of the need to increase the insulin sensitivity in patients with the metabolic syndrome and thus to correct the dyslipidaemia which is considered to cause the accelerated progress of atherosclerosis. However, currently this is not a universally accepted diagnosis with well-defined pharmacotherapeutic indications.

Co-pending PCT application No. PCT/GB02/05743 discloses compounds of formula A 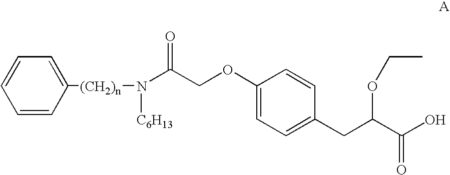
wherein n is 1 or 2 and pharmaceutically acceptable salts, solvates, crystalline forms and prodrugs thereof are highly potent PPARα modulators. A process for the preparation of such compounds is described which comprises reacting the S-enantiomer of a compound of formula B 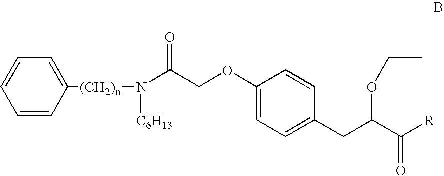
in which n is as previously defined and R represents a protecting group for a carboxylic hydroxy group as described in the standard text “Protective Groups in Organic Synthesis”, 3rd Edition (1999) by Greene and Wuts, with a de-protecting agent.

Compounds of formula B may be prepared by reacting the S-enantiomer of a compound of formula C 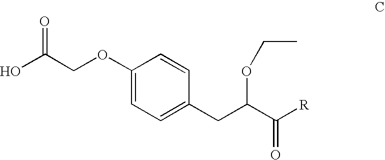
in which R is as previously defined with a compound of formula D 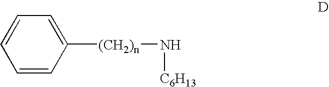
in which n is as previously defined in an inert solvent, for example dichloromethane, in the presence of a coupling agent, for example a carbodimide, eg 1-(3-dimethylaminopropyl)-3-ethylcarbodiimide, and optionally in the presence of a catalyst, for example a basic catalyst, eg 4-dimethylaminopyridine, at a temperature in the range of −25° C. to 150° C. An improved process for the preparation of compounds of formula A has now been found.

### DESCRIPTION OF THE INVENTION

The present invention provides a process for the preparation of a compound of formula I 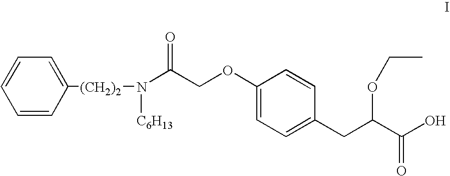
in which a compound of formula II 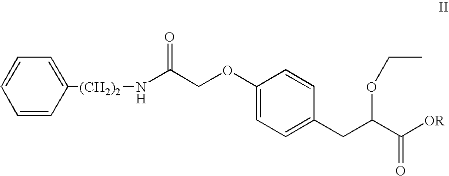
in which R is H or OR represents a protecting group for a carboxylic hydroxy group is reacted with a compound of formula III
C₆H₁₃X III
wherein X is a leaving group, in the presence of a base in the presence of an inert solvent at a temperature in the range −25° C. to 150° C. and optionally, when OR represents a protecting group, removal of the protecting group.

One particular embodiment of the invention provides a process for the preparation of a compound of formula I 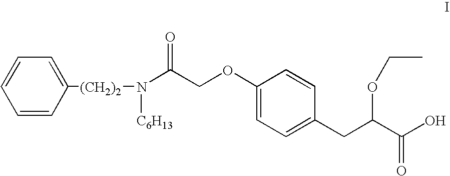
comprising reacting a compound of formula IV 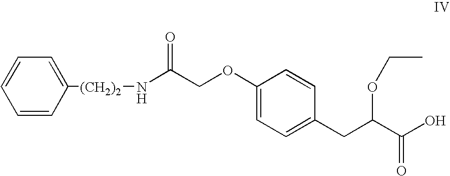
with a compound of formula III
C₆H₁₃X III
wherein X is a leaving group in the presence of a base in the presence of an inert solvent at a temperature in the range −25° C. to 150° C.

The protecting groups OR and deprotecting agents are described in the standard text “Protective Groups in Organic Synthesis”, 3^{rd} Edition (1999) by Greene and Wuts, which is herein incorporated by reference. Suitable protecting groups include where OR represents a C₁₋₆alkoxy group eg ethoxy group or an arylalkoxy group eg benzyloxy. In particular, when OR represents a C₁₋₆alkoxy group eg ethoxy group or an arylalkoxy group eg benzyloxy, such that COOR represents an ester then such esters may be reacted with a de-protecting agent e.g. a hydrolysing agent, for example lithium hydroxide in a mixture of THF and water, at a temperature in the range of 0-100° C.

Suitable bases include potassium hydroxide, sodium hydroxide, lithium hydroxide, sodium hydride, potassium tert-butoxide, cesium carbonate, potassium carbonate, or sodium carbonate particularly potassium hydroxide.

Suitable inert solvents include dimethyl sulphoxide, N,N-dimethylformamide, N-methylpyrrolidone or toluene or mixtures thereof, particularly dimethyl sulphoxide.

Suitably X represents bromo, chloro, OSO₂CH₃, OTosyl, OSO₂CF₃, OC(O)OR, OP(O)(OR)₂ or OSO₂OR. Particularly X is chloro or bromo.

Optionally a phase transfer catalyst may be used for example an alkylammonium salt for example a tetraalkylammonium halide salt eg tetrabutyl ammonium bromide.

Compounds of formula II in which R is H (or compound IV) may be prepared by reacting a compound of formula II 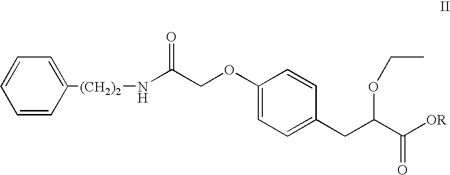
in which OR represents a protecting group for a carboxylic hydroxy group with a de-protecting agent. In particular, OR represents a C₁₋₆alkoxy group eg ethoxy group or an arylalkoxy group eg benzyloxy, such that COOR represents an ester. Such esters can be reacted with a de-protecting agent e.g. a hydrolysing agent, for example lithium hydroxide in a mixture of THF and water, at a temperature in the range of 0-100° C.

Compounds of formula II in which OR represents a protecting group for a carboxylic hydroxy group may be prepared by reacting a compound of formula V 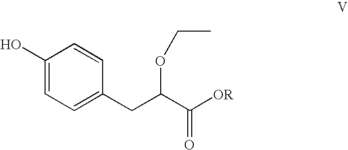
in which OR is as previously defined with a compound of formula VI 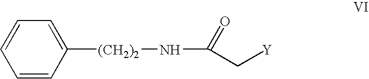
in which Y represents a leaving group, for example halo, particularly chloro, in an inert solvent, for example acetonitrile, acetone, methyl isobutylketone, N-methylpyrrolidone, toluene, toluene/water, ethanol or isopropylacetate in the presence of a base, for example potassium carbonate, sodium hydroxide or triethylamine, at a temperature in the range of 0° C. to 150° C. Optionally a catalyst may be used for example iodide or a quartenary ammonium salt, particularly sodium iodide or tetra-n-butylammonium-iodide, -bromide, -acetate or -hydrogensulphate.

It is believed that the compound of formula II in which R is H, namely (2S)-2-ethoxy-3-(4-{2-oxo-2-[(2-phenylethyl)amino]ethoxy}phenyl)propanoic acid (compound IV), is novel and is herein claimed as a further part of the present invention. This compound has the advantage of being a solid and therefore offers an opportunity for purification and isolation during the reaction sequence if desired. Also claimed herein is a compound of formula II in which OR represents a protecting group for a carboxylic hydroxy group in particular OR represents for example a C₁₋₆alkoxy group eg methoxy,ethoxy or propoxy or an arylalkoxy group wherein aryl is phenyl optionally substituted by C₁₋₆alkyl, C₁₋₆alkoxy or halo, eg benzyloxy, for example compound VII

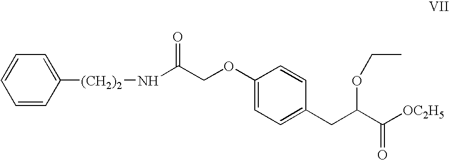

In another aspect the present invention provides a process for preparing a pharmaceutically acceptable salt of the compound of formula I comprising reacting the acid obtained by one of the processes of the present invention with a base, optionally in the presence of a solvent and isolating the salt.

Preferably the compound of formula I prepared by the process is the (2S)-enantiomer. Similarly the preferred compounds of formulae Il and VII are the (2S)-enantiomers.

### EXAMPLES

¹H NMR and ¹³C NMR measurements were performed on a Varian Mercury 300 or Varian UNITY plus 400, 500 or 600 spectrometers, operating at ¹H frequencies of 300, 400, 500 and 600 MHz, respectively, and at ¹³C frequencies of 75, 100, 125 and 150 MHz, respectively. Measurements were made on the delta scale (δ).

Unless otherwise stated, chemical shifts are given in ppm with the solvent as internal standard.

### ABBREVIATIONS

DMSO dimethyl sulfoxide
THF tetrahydrofuran
t triplet
s singlet
d doublet
q quartet
m multiplet
bs broad singlet
dm doublet of multiplet
bt broad triplet
dd doublet of doublet
dq doublet of quartet

### Example 1

### (2S)-2-ethoxy-3-(4-{2-[hexyl(2-phenylethyl)amino]-2-oxoethoxy}phenyl)propanoic acid

a) Phenethylamine (30.0 g) was treated with 6M aqueous sodium hydroxide (61.5 ml) in toluene (100 ml). A solution of chloroacetyl chloride (28.0 g) in toluene (50 ml) was added under temperature control. After complete reaction, the reaction slurry was warmed until a complete solution was obtained, and the water-phase was removed. The organic phase was washed with aqueous hydrogen chloride and water. The resulting toluene phase was reduced by evaporation and diisopropylether was added to the toluene solution. The solution was cooled and 1-chloro-N-phenethylacetamide (42.3 g) was collected by filtration, washed and dried. The product was analysed by LC (99.8 area %) and NMR.

¹H NMR δ_{H}(400 MHz, CDCl₃): 2.88 (t, 2H), 3.60 (dd, 2H), 4.05 (s, 2H), 6.62 (bs, 1H), 7.19-7.58 (m, 5H).

b) A mixture of potassium carbonate (31.5 g), 1-chloro-N-phenethylacetamide (15.0 g), ethyl (2S)-2-ethoxy-3-(4-hydroxyphenyl)propanoate (18.1 g) (see WO 99/62871) and acetonitrile (150 ml) was stirred and brought to the boil under reflux. After complete reaction, the mixture wass cooled and the inorganic salts were filtered off and washed with acetonitrile. The remaining solution was reduced by distillation and the product was crystallised from ethyl acetate and hexanes. Ethyl (2S)-2-ethoxy-3-(4-{2-oxo-2-[(2-phenylethyl)amino]ethoxy}phenyl) propanoate (24.5 g) was collected by filtration, washed and dried. The product was analysed by LC (98.6 area %) and NMR.

¹H NMR δ_{H}(400 MHz, CDCl₃): 1.18 (t, 3H), 1.26 (t, 3H), 2.86 (t, 2H), 2.96-3.01 (m, 2H), 3.37 (dq, 1H), 3.58-3.68 (m, 3H), 4.00 (dd, 1H), 4.20 (q, 2H), 4.47 (s, 2H), 6.65 (bs, 1H), 6.79 (dm, 2H), 7.14-7.36 (m, 7H).

c) A solution of ethyl (2S)-2-ethoxy-3-(4-{2-oxo-2-[(2-phenylethyl)amino]ethoxy}-phenyl)propanoate (36.0 g) in THF (270 ml) was added to a solution of lithium hydroxide (6.51 g) dissolved in water (360 ml). The mixture was stirred at room temperature. After complete reaction, the mixture was evaporated under reduced pressure to remove THF. After evaporation, the reaction mixture was cooled to room temperature and acidified with hydrochloric acid. The acidified product was extracted with ethyl acetate. The ethyl acetate solution was washed with water and evaporated to a reduced volume. The product was crystallised from ethyl acetate and diisopropyl ether. (2S)-2-Ethoxy-3-(4-{2-oxo-2-[(2-phenylethyl)amino]ethoxy}phenyl)-propanoic acid (28.0 g) was filtered off and washed with diisopropyl ether and dried under vacuum.

¹H NMR δ_{H}(400 MHz, CDCl₃): 1.20 (t, 3H), 2.85 (t, 2H), 3.00 (dd, 1H), 3.10 (dd, 1H), 3.46 (dq, 1H), 3.56-3.71 (m, 3H), 4.07 (dd, 1H), 4.45 (s, 2H), 6.68 (bs, 1H), 6.78 (dm, 2H), 7.10-7.38 (m, 7H).

d) Dimethylsulfoxide (DMSO) (2750 mL), potassium hydroxide powder (244 g) and (2S)-2-ethoxy-3-(4-{2-oxo-2-[(2-phenylethyl)amino]ethoxy}phenyl)propanoic acid (250 g) were stirred at approximately 18° C. for ca 20 minutes. 1-Bromohexane (344 g=292 mL) was added over 2.5 hours. The reaction mixture was stirred for approximately 10 minutes. Diisopropyl ether (1000 mL) was added followed by filtration, extraction and separation of the mixture. The DMSO layer was further extracted with diisopropyl ether (2×1000 mL). The DMSO layer was acidified with 4M HCl(aq) (950 mL). Diisopropyl ether (3000 mL) and water (2500 mL) were added followed by extraction. The layers were separated (pH˜2 of aq layer) and the diisopropyl ether layer was washed with water (2500 mL). The diisopropyl ether layer was concentrated in vacuo to a clear, very viscous oil. Yield 317 g, assay 88.1%, corrected yield 91.1%, LC-purity 97.2%, e.e. 97.8%. LC-purity and kiral LC in accordance with reference sample.

¹H NMR δ_{H}(400 MHz, CDCl₃): 0.75-0.85 (m, 3H), 1.10 (t, 3H), 1.14-1.29 (m, 6H), 1.40-1.55 (m, 2H), 2.76-2.93 (m, 3H), 2.97-3.06 (m, 1H), 3.06-3.14 and 3.28-3.43 (2m, 3H, rotamers), 3.45-3.58 (m, 3H), 3.98 (m, 1H), 4.32 and 4.59 (2s, 2H, rotamers), 6.68 and 6.80 (2dm, 2H, rotamers), 7.02-7.31 (m, 8H).

1. A process for the preparation of a compound of formula I 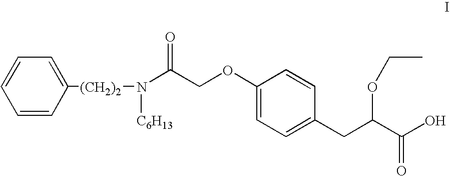
in which a compound of formula II 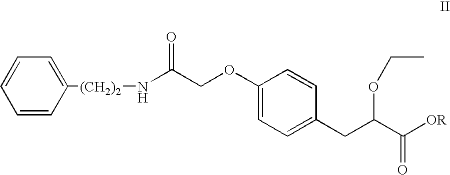
in which R is H or OR represents a protecting group for a carboxylic hydroxy group is reacted with a compound of formula III
C₆H₁₃X III
wherein X is a leaving group, in the presence of a base in the presence of an inert solvent at a temperature in the range −25° C. to 150° C. and optionally, when OR represents a protecting group, removal of the protecting group.

2. A process for the preparation of a compound of formula I 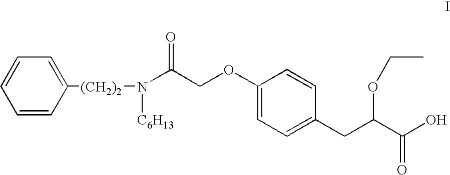
comprising reacting a compound of formula IV 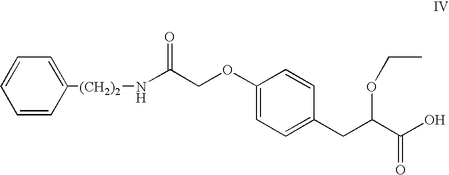
with a compound of formula III
C₆H₁₃X III
wherein X is a leaving group in the presence of a base in the presence of an inert solvent at a temperature in the range −25° C. to 150° C.

3. A compound of formula II 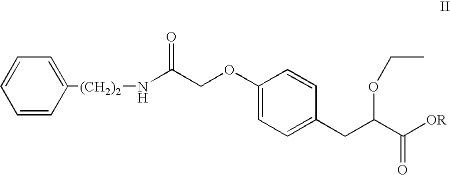
in which OR represents a protecting group for a carboxylic hydroxy group.

4. A compound according to claim 3 in which OR represents a C₁₋₆alkoxy group.

5. A compound according to claim 3 which is the 2S enantiomer.

6. The compound (2S)-2-ethoxy-3-(4-{2-oxo-2-[(2-phenylethyl)amino]ethoxy}phenyl)-propanoic acid.

7. A process according to claim 1 to produce the (2S) enantiomer of the compound of formula I by using the 2S enantiomer of the compound of formula II.

8. A compound according to claim 4 which is the 2S enantiomer.
